SEQUENCE LISTING (1 sequence)

SEQ ID NO: 1 - DNA, 1645 bp, Scenedesmus species Novo
  ref <308>: DATABASE ACCESSION NUMBER: Genbank HQ900842
  ref <309>: DATABASE ENTRY DATE: 2011-03-06
  ref <313>: RELEVANT RESIDUES IN SEQ ID NO: (1)..(1645)
tgcttatactgtgaaactgcgaatggctcattaaatcagttatagtttatttggtggtaccttcttactcggaataaccgtaagaaaattagagctaatacgtgcgtaaatcccgacttctggaagggacgtatatattagataaaaggccgaccgggctctgcccgacccgcggtgaatcatgatatcttcacgaagcgcatggccttgtgccggcgctgttccattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcctgatacggggaggtagtgacaataaataacaataccgggcatttcatgtctggtaattggaatgagtacaatctaaatcccttaacgaggatccattggagggcaagtctggtgccagcagccgcggtaattccagctccaatagcgtatatttaagttgttgcagttaaaaagctcgtagttggatttcgggtgggtttcagcggtccgcctatggtgagtactgctgtggccttccttactgtcggggacctgcttctggggcttcattgtccgggacagggattcggcatggttactttgagtaaattagagtgttcaaagcaggcttacgcccgtgaatactttagcatggaataacatgataggactctgccctattctgttggcctgtaggagtggagtaatgattaagaggaacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattggcggacgtttttgcatgactccgtcagcaccttgagagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgtcttgtcaggttgattccggtaacgaacgagacctcagcctttaaatagtcacggtcgctttttgcggctggtctgacttcttagagggacagttggcgtttagtcaacggaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgattacgtccctgccctttgtacacaccgcccgtcgctcctaccgattgggtgtgctggtgaagtgttcggattggcaattgaaggtggcaacaccgtcgatgccg

(19) United States
(12) Patent Application Publication
(10) Pub. No.: US 2015/0299646 A1
(43) Pub. Date: Oct. 22, 2015

(54) CULTURED EXTREMOPHILIC ALGAE SPECIES NATIVE TO NEW MEXICO

(71) Applicant: STC.UNM, Albuquerque, NM (US)

(72) Inventors: Ravi Venkata Durvasula, Albuquerque, NM (US); Subba Rao Durvasula, Dartmouth (CA); Annabeth Fieck, Albuquerque, NM (US); Ivy Hurwitz, Albuquerque, NM (US)

(21) Appl. No.: 14/698,481

(22) Filed: Apr. 28, 2015

Related U.S. Application Data
(63) Continuation-in-part of application No. 13/723,687, filed on Dec. 21, 2012, now abandoned.
(60) Provisional application No. 61/579,120, filed on Dec. 22, 2011.

(51) Int. Cl. C12N 1/12; C02F 3/32; A01N 65/03

(52) U.S. Cl. CPC C12N 1/12; A01N 65/03; C02F 3/322

(57) ABSTRACT

Provided herein is an extremophile green alga designated as Scenedesmus species Novo, from Jemez warm water springs, New Mexico. Sequencing 18S rDNA confirmed the alga as a new species. It is capable of producing high levels of microalgal biomass in wastewater under harsh ambient climatic conditions, and of yielding high levels of lipids and carotenes. Cultures in TAP medium at 24±1° C. at continuous light (132-148 μmol photons m⁻²s⁻¹) attained peak biomass levels 27.4×10⁶ cells ml⁻¹, 49.11 μg ml⁻¹ chlorophyll α, 24.93 μg ml⁻¹ carotene on the seventh day and a division rate of 0.54 day⁻¹. High levels of biomass were sustained in sterilized and unsterilized municipal wastewater, enriched with 1% TAP nutrients or unenriched. The microalga is useful in the production of biofuels, fertilizers, dietary nutrients, pharmaceuticals, polymers, biofilters to remove nutrients and other pollutants from wastewaters, in space technology, and laboratory research systems.

### CROSS-REFERENCE TO RELATED APPLICATIONS

This application is a continuation-in-part application claiming priority to U.S. patent application Ser. No. 13/723,687 filed Dec. 21, 2012, of identical title, and U.S. Provisional Patent Application Ser. No. 61/579,120 filed Dec. 22, 2011, each of which is incorporated herein by reference in its entirety.

### STATEMENT OF GOVERNMENT SUPPORT

This invention was made at least in part with Government support from the Department of Veterans Affairs. The Government has certain rights in the invention.

### BACKGROUND OF THE INVENTION

Despite significant investment in research and development, commercial viability of algal-derived biofuels remains a future prospect. Costs of mass algal culture, including commercially available nutrient stocks such as f/2 media cost $25/liter unpredictability of algal stocks (see Ravi, et al. 2013), high costs of algal concentration and extraction of products and limited choices for algal stocks all contribute to the untenable costs of algal biofuel—in excess of $17 per gallon—and the limited use of this energy source in the open market (Ravi, et al., 2013).

Sewage sludge is rich in various nutrients. Analyses of sewage sludge samples from 6 north-central states in USA yielded median concentrations as follows: N, 4.2; P, 3.0; K, 0.3%; Pb, 540; Zn, 1,890; Cu, 1,000; Ni, 85; and Cd, 16 mg/kg, and for aerobically treated sludges: N, 4.8; P, 2.7; K, 0.4%; Pb, 300; Zn, 1,800; Cu, 970; Ni, 31; and Cd, 16 mg/kg (Sommers 1977). Sewage is a source of nutrients both organic and inorganic that sustain algal growth. Growth of these algae could result in blooms either benign or toxigenic which could have serious environmental and societal impacts.

Microalgae synthesize organic material from inorganic material via photosynthesis which can be expressed as: 6CO₂+6H₂O+light 8 photons [symbol] C₆14₁₂O₆+6O₂↑

During photosynthesis, microalgae assimilate macronutrients (N, P, S) and the trace elements (Fe, Zn, Mn) as expressed below:

106CO₂+16NO₃+PO₄+SO₄+10⁻²Fe+4×10⁻³Zn+4×10⁻⁴Mn [symbol] (C106H263O110N16PS)+138O₂↑

Organic matter (C106 H263 O110 N16 PS) and oxygen are the two most important products.

Traditionally microalgal cultures both marine and freshwater are grown in media with high concentrations of nutrients (Table 1A) that are several orders of magnitude higher than those in the marine environment (Table 2A):

Range of nutrients in culture media

| | | Marine (24)* | Freshwater(26) |
|---|---|---|---|
| Macro- | PO₄ | 0.5 μM-10 mM | 0.73 μM-2 mM |
| nutrients | N₂ | 1 μm-9.9 mM | 0.1 μm-17.6 μM |
| | Si | 10 μM-0.7 mM | 12.5 μM-30 mM |
| Trace | Fe | 1 μM-1.8 mM | 0.72 μM-17.9 μM |
| metals | Cu | 0.24 nM-0.063 mM | 0.4 nM-6.29 μM |
| | Co | 0.063 nM-0.04 mM | 8.1 nM-0.68 μM |
| | Zn | 0.3 nM-3.48 mM | 0.8 nM-30.7 μM |
| | Mn | 0.21 nM-1.4 mM | 0.18 μM-7.28 μM |
| Vitamins | B₁₂ | 3.69 pM-7.4 nM | 0.738 pM-1.84 nM |
| | Thiamine | 0.3 nM-3 mM | 73.8 pM-0.148 μM |
| | Biotin | 3.27 nM-0.2 μM | 0.41 nM-10.2 nM |

TABLE 2A
Range of selected major nutrients used in algal cultures

| Enrichment | μM in Oceans | |
|---|---|---|
| ▾ | (Turekian 1968) | Range in cultures |
| Phosphorus | 2.84 | 0.5 μM-10 mM |
| Nitrogen | 1106 | 1 μM-9.9 mM |
| Silica | 103 | 10 μM-500 μM |
| Iron | 0.06 | 1.0 μM-1.8 mM |
| Copper | 0.014 | 0.24 nM-0.063 mM |
| Cobalt | 0.07 | 0.063 nM-0.04 mM |
| Zinc | 0.08 | 0.3 nM-76.5 μM |
| Manganese | 0.07 | 0.207 nM-1.4 mM |
| Vitamin B₁₂ | — | 3.69 pM-7.4 nM |
| Thiamine HCl | — | 0.3 nM-3 mM |
| Biotin | — | 3.27 nM-0.2 μM |

Cultures of microalgae have the potential for bioremediation because of their ability to assimilate and bioaccumulate several nutrients. Under defined culture conditions of temperature (25-27° C.) and fluorescent light with a light:dark photoperiod of 15 h:9 h, the microalgae Tetraselmis chuii and Nannochlopropsis sp. have been utilized for removal of nutrients in recirculation aquaculture systems in waste water (Sirakov and Velichikova 2014). N. oculata removed 78.4% of total nitrogen, 92% of nitrate and 42.3% phosphate. Utilizing bacterial-biofilm bioreactors higher rates of removal i.e. 91±3%, 70±8% and 85±9% for carbon, nitrogen and phosphorus, respectively, are also possible (Posadas et al. 2013). Chlorella vulgaris and algae taken from Pleasant Hill Lake, Ohio grown under defined conditions were used for bioremediation of wastewater laden with nitrogen, phosphorous, chromium (Cr (VI)) and cadmium (Cd (II) (Saikumar 2014).

Most of the microalagal cultures are raised under defined conditions of temperature and under a bank of growlux fluorescent lights which escalate production costs (Table 3A). The key to successful bioremediation would be to raise microalgal cultures in waste water under ambient conditions of light and temperature. Incidentally this would remove the nutrients from the wastewater via bioaccumulation by microalgae.

TABLE 3A
Production costs of marine microalgae

| | | Production cost US$ | |
|---|---|---|---|
| Taxa | Nature of culture | per kg-l dry weight | Reference |
| T-iso, Skeletonema sp. | Tanks | 1000 | Bennemann 1992 |
| Pavlova lutheri, | | | |
| Nannochloropsis sp. | | | |
| Tetraselmis suecica | Batch | 300 | Coutteau and Sorgeloos 1992 |
| Various diatoms | Continuous flow cultures 240 m³ | 167 | Walsh et al 1987 |
| Nannochloropsis sp | Photobioreactors | 100 | Chini Zittelli et al. 1999 |
| Monospecific algal culture | Indoors or in a green house | 120-200 | De Pauw et al. 1984. |
| | Outdoor culture | 4-20 | De Pauw and Persoone 1988 |
| | Tank culture 450 m³ | | Donaldson 1991 |
| Algal biomass | Photobioreactors and Fermentors | 11.22 | Behrens 2005 |
| (Autotrophic) | | | |
| Algal biomass | Photobioreactors and Fermentors | 2.01 | Behrens 2005 |
| (Heterotrophic) | | | |
| Tetraselmis suecica | Fermentors | 10 | Day et al. 1991 |
| Cyclotella cryptica | | 170 | Gladue and Maxey 1994 |
| Nitzschia alba | | 12 | ″ |
| Chlorella sp. | | 160 | ″ |
| Cyclotella | | 600 | ″ |
| | | | Barclay et al. 1994 |
| | | | De Swaaf et al. 1999 |
| Chlorella sp. | | | |
| Crypthecodium cohnii | | | |
| Schizochytrium sp | | | |
| Induced blooms of marine | | 4-23 | De Pauw et al. 1984 |
| phytoplankton species | | | |
| Wastewater- grown | | 0.17-0.29 | De Pauw et al. 1984 |
| microalgae | | | |

### SUMMARY OF THE INVENTION

Provided herein is an isolated and purified new microalgal species designated Scenedesmus species Novo and progeny thereof. The alga was collected at latitude 35.769 and longitude 106.692. It is capable in culture including TAP medium of producing a biomass of about 10.41×10⁶ cells per ml and at least about 4 μg per ml, for example, about 4.18 to about 4.5 μg per ml, of carotene under outdoor growth conditions comprising temperatures reaching 40° C. or higher.

The new microalgal species has an 18S ribosomal RNA gene sequence [SEQ ID NO:1] at least about 99% to about 100% identical to SEQ ID NO:1, and about 98% identical to algal species G24 (38).

In embodiments, the alga is capable of producing up to at least about 3.58 pg per cell of carotenes under indoor growth conditions. The term “up to at least about” as used with respect to a numerical value herein refers to a value seen at any point on a graph of such values over time.

The cultures can be cultivated in sewage/wastewater at ambient temperatures of up to at least about 40° C. In embodiments the cultures are cultivated at temperatures above 40° C., for example between about 40° C. and about 100° C., or between about 40° C. and about 80° C., or between about 40° C. and about 60° C., or between about 40° C. and about 50° C. As used herein, the term “extremophilic microalgae” refers to thermophilic microalgae capable of growth at such temperatures. The microalga of the present invention may be used to treat sewage/wastewater (it is a freshwater microalga) and provides high production of hydrocarbons, especially carotenoids and provides bioremediation of the sewage/wastewater making the treated sewage/wastewater far easier to further process in water treatment plants to clean water.

Cultivated in water enriched with growth-promoting nutrients such as those of TAP medium, at ambient room temperatures (e.g., about 20° C. to about 26° C.), cultures of this microalga are capable of producing an average lipid content of between about 63 pg per cell and about 95 pg per cell. Cultures grown in enriched TAP medium indoors can have a chlorophyll α content up to between about 20 and about 49 μg per ml, and a carotene content up to about 10 to about 24 or about 25 μg per ml.

In embodiments, grown outdoors in wastewater at temperatures that reach 40° C. or higher, in a TAP medium, such cultures can have a lipid content of between about 16.7 and 81.4 pg per cell, a chlorophyll α content up to about 5.8 μg ml⁻¹, and a carotene content over 4 μg ml⁻¹, e.g., about 4.18 to about 4.5 μg ml⁻¹.

The cultured algae are circular and can be single cells and/or clumps of up to about 360 cells which drop to the bottom of the vessel containing the culture, thus making it easy to harvest the cells. Harvested algal biomass produced by the microalgae can be dried to a mass having a water content less than about 5%.

A method for culturing and harvesting extremophilic microalgae is also provided herein. The method comprises preparing a growth medium composition comprising said extremophilic microalgae and water (including sewage/wastewater) comprising nutrients capable of enhancing growth of the microalgae; allowing the microalgae to proliferate in the composition under ambient outdoor conditions comprising intervals of ambient temperatures of at least about 40° C. and ambient light of up to about 1400 to about 1600 watts; and dewatering the composition and recovering and drying it to obtain an algal biomass comprising the microalgae and less than about 5% water content. This same method or a similar method may be readily adapted for use on sewage/municipal wastewater for bioremediation of the sewage/wastewater, making it far more easy to process in water treatment plants.

The dewatering step can be performed in a micro solid-liquid separation system such as one from AlgaeVenture Systems, Marysville, Ohio. In preferred embodiments, the extremophilic microalgae in the growth composition are Scenedesmus species Novo. In embodiments, the growth composition also comprises wastewater, often municipal wastewater (sewage). In embodiments, the nutrients in the growth composition are selected from the group consisting of TAP medium components, selenium, boron and iron. The wastewater can be sterilized urban or agricultural wastewater or nonsterilized urban or agricultural wastewater. The wastewater may also be industrial or residential wastewater, often residential wastewater or a combination of residential (municipal) wastewater and industrial wastewater. Any wastewater in which the microalgae of the present invention may grow represents a source of nutrients which be converted by the microalgae of the present invention. Thus, the present invention may be used to convert sewage/wastewater to useful lipids and hydrocarbons, especially including carotenes in high concentrations under conditions in which most microalgae are incapable because of the extreme conditions of certain embodiments of the present invention and to bioremediate the sewage/wastewater to make it less dangerous and more easy to process to clean water (e.g. in water treatment plants).

In another embodiment hereof, a method for culturing and harvesting extremophilic microalgae is provided comprising: preparing a growth composition comprising the extremophilic microalgae and water, which often constitutes sewage/municipal wastewater and often further comprises TAP medium components in amounts sufficient to enhance growth of said microalgae; allowing the microalgae to proliferate in said composition at room temperatures, such as temperatures of about 23° C. to about 25° C., or higher; and dewatering and drying the composition and recovering an algal biomass comprising the microalgae and less than about 5% water content. In embodiments of this method, the microalgae are Scenedesmus species Novo. In embodiments, the growth composition comprises sewage/wastewater and further includes the components of TAP medium and optionally further components such as selenium, boron and iron, among others.

A method of inhibiting growth of a microorganism is also provided herein. The method comprises contacting cells of the microorganism with an extract of Scenedesmus species Novo. The microorganisms can be bacteria, viruses, parasites, or fungi.

Applicants have isolated an extremophile green alga, Scenedesmus species Novo, with unique growth and biochemical characteristics, from Jemez warm water springs in New Mexico. Sequencing 18S rDNA confirmed the alga as a new species. Cultures in TAP medium at 24±1° C. at continuous light (132-148 μmol photons m⁻²s⁻¹) attained peak biomass levels of 27.4×10⁶ cells ml⁻¹ with a division rate (k) of 0.54 day⁻¹, and yielded 49.11 μg chlorophyll α ml⁻¹ and 24.93 μg carotene ml⁻¹ on the 7th day high levels of biomass were sustained in sterilized or unsterilized municipal wastewater, either enriched with 1% TAP nutrients or unenriched. Under outdoor conditions (6524-7360 μmol photons m⁻²s⁻¹ and ˜40° C.), high levels of biomass (10.41×10⁶ cells ml⁻¹), and yields of 8.92 μg chlorophyll α ml⁻¹, and 4.18 μg carotene ml⁻¹ were sustained. Lipids in cells raised in TAP under controlled, less severe conditions ranged from 63 to 94.3 pg cell⁻¹, and in outdoor wastewater 16.7 to 81.4 pg cell⁻¹, which are higher than those previously reported in the literature. In cultures raised in TAP in outdoor waste water, lipid (% of cell dry weight) ranged from about 15% to about 74%, substantially higher than previous literature values. Total carotenoids ranged between 0.37 and 3.58 pg cell⁻¹. Thus, in preferred embodiments, the microalgae may be used in freshwater, making it particularly useful to treat sewage/municipal wastewater to produce high concentrations of hydrocarbons, especially carotenes, and can be used to make the sewage/wastewater far less polluted and more amenable and easier to process to clean water. Moreover, the microalgae may be used at varying temperatures from room temperature to temperatures of up to at least about 40° C. to about 100° C. or more (depending on the pressure of the medium in which the microalgae is grown).

Because of its ability to produce high levels of microalgal biomass in wastewater under harsh ambient climatic conditions and yield of high levels of lipids and carotenes, mass cultivation of Scenedesmus species Novo is useful in many biotechnological applications. Because of the extremophilic nature of Scenedesmus species Novo, this microalgae is particularly suited for industrial use because it can tolerate high temperatures which often will cause difficultires for other microalgae and microorganisms in culture. Accordingly, the microalgae of the present invention, because of its extremophilic stability and its ability to grow in fresh water culture (making it useful for sewage/wastewater treatment compared to salt water species) and produce high concentrations of lipids and/or carotenoids in culture, providing methods of the present invention that are more reliable, resilient and cost effective than prior art approaches.

### BRIEF DESCRIPTION OF THE DRAWINGS

FIG. 1 shows a con-focal image of Scenedesmus species Novo.

FIG. 2 shows the growth of Scenedesmus species Novo in TAP and BG11 media.

FIG. 3 shows temporal variations in cellular pigments in indoor cultures.

FIG. 4 shows temporal variations in cellular pigments in outdoor cultures.

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

The singular forms “a,” “an,” and “the,” include plural referents unless expressly and unequivocally limited to one referent. Thus, for example, reference to “an inhibitor” can include two or more different compounds. As used herein, the term “include” and its grammatical variants are intended to be non-limiting, such that recitation of items in a list is not to the exclusion of other like items that can be substituted or other items that can be added to the listed items.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

“Wastewater” includes, but is not limited to, contaminated surface and subsurface runoff water from storm events and acid mine drainage, coking wastewater generated in the high-temperature carbonization of raw coal, coal gas purification and refining process of chemical products, raw untreated sewage wastewater having a significant concentration of waste solids, water comprising any number of pollutants found in urban, residential and agricultural settings around the world, storm runoff which picks up a wide variety of contaminants as it flows across the surface and then into private and public waters, runoff that flows across roads and parking lots and that picks up oil, grease and metals from automobile discharges, or that picks up nitrate and phosphate from fertilized lawns and golf courses, or that picks up organic waste, herbicides and pesticides from agricultural sites, or that picks up grit and colloidal particles from all of these locations, water sources impacted by mining, which include surface and subsurface flows, water containing a wide variety of pollutants related to hydrologic fracturing for natural gas as well as acidified mine drainage water carrying heavy loads of dissolved metals and waters such as streams, rivers and lakes, aquifers and groundwater containing any contaminant. In certain preferred embodiments according to the present invention, the wastewaster used is urban wastewater, often industrial or municipal/domestic wastewater or a combination of municipal/domestic wastewater and industrial wastewater (from standard sewage runoff). The use of sewage/municipal wastewater is preferred. Domestic wastewater includes wastewater from residential settlements and services which originates predominantly from the human metabolism and from household activities.

The term “microalgae” refers collectively to unicellular organisms that have photosynthetic pigments and are photosynthesized. Microalgae can grow in the presence of a suitable amount of light and dissolved nutrients and can be utilized in various applications, including the production of biomass and biofuel and the improvement of atmospheric and aquatic environments. The preferred microalgae for use in the present invention is Scenedesmus species Novo.

Microalgae have several advantages as feedstock to land-based biofuels. They are renewable and amenable for mass cultivation on nonarable land; they can be a source of significant quantities of lipids; they act as a source of value-added co-products; they can be used for bioremediation; and they are capable of sequestering carbon. Microalgal biomass can yield between 58,700 and 90,000 liters of biodiesel per hectare per year (1,2,3). Biofuels contribute to ˜2% of global transport fuel today but are predicted to increase to 27% by the year 2050 (4). For biotechnological applications, sustenance and steady supply of algal biomass are required, which is feasible by mass cultivation of algae. Only a small percentage of the 17,500 microalga species are cultured and about 50 have been screened for their utility—mostly in biofeed, with only a few having been identified as useful for biofuel. Most of the algal isolates are from temperate waters and are grown in defined sterile media under controlled conditions of temperature and light, which collectively escalate biomass production costs to as high as $7.32 per kg of algal biomass and $24.60 per liter algal oil (5).

Microalgae characterized as extremophiles remain least studied. Extremophile algae can readily adapt to exacting local physicochemical conditions, and manifest biochemical and physiological responses such as the production of carotenoids, as in Dunaliella salina (6). The extremophile diatom Nitzschia frustula and the green alga Chlamydomonas plethora isolated from the semiarid harsh climate of the Arabian Gulf (7) have high division rates, carbon assimilation rates (18.1 22.8 to mg C per mg chlorophyll α per hour) approaching their theoretical maxima as well as yielding levels of acids and leucine, lysine, glutamic acid and arginine that make them valuable in biotechnological applications.

Reported here are observations on Scenedesmus species Novo, an extremophile green alga isolated by us from Jemez Springs, N. Mex. This alga grows well in urban wastewater under ambient conditions of light and temperature in New Mexico, yields considerable quantities of lipids and carotenoids and is especially useful for producing algal biofuel.

The invention is illustrated further in the following non-limiting examples.

### Example 1

### Cultured Cells of Scenedesmus Species Novo

Several samples of water were collected from Jemez warm water springs (latitude 35.769 and longitude 106.692) and enriched with nutrients f/50, f/10 (28) and TAP media. Samples were incubated at 24±1° C. at continuous 132-148 μmol m⁻²s⁻¹ light supplied by cool white fluorescent lights. Using repeated serial dilution techniques algal cultures were established. Pure cultures were based on isolates established by streaking on agar plates. Agar slants were based on enrichments with f/50, BG11 and TAP media. Utilizing usual sterile culture techniques, colonies were isolated and gradually scaled up into BG11 (29) and modified TAP medium (30). TAP medium based on enrichment with 10 ml each of triacetate stock, nutrient stock, phosphate buffer and trace elements supported excellent growth. Trace element enrichment follows the formula as described in Hunter (1950) (31).

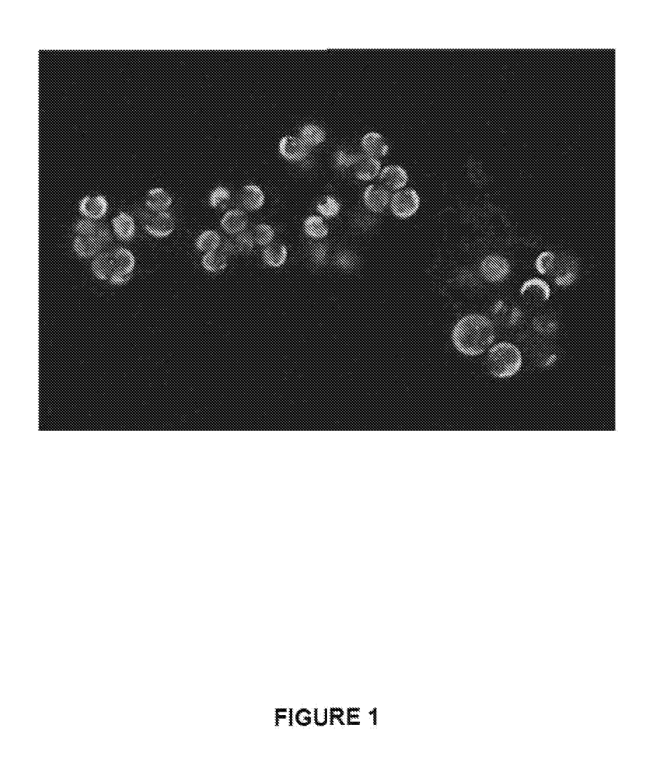

Cultured cells of Scenedesmus species Novo were circular, either singular or in clumps up to 356 cells and did not have any spines. Cells were non-motile, enveloped in mucilage (FIG. 1). Well-mixed cells left in culture flasks sank to the bottom in a couple of minutes which is advantageous in harvesting the biomass.

All algae samples collected from this location exhibited similar properties and were considered to be samples of the same species.

### Cultures in Defined Media

All growth experiments were done in triplicate. Samples were incubated at 24±1° C. at continuous 132-148 μmol m⁻²s⁻¹ light supplied by fluorescent lights, or were incubated over the terrace of a building under natural light (1400-1600 watts m-², equivalent to 6524-7360 μmol m⁻²s⁻¹), and ˜40° C. Suitable aliquots were drawn from each culture aseptically for enumeration, chlorophyll a and carotenoid determinations. Direct counts were made on the samples using an Improved Neubauer haemocytometer.

### Division Rates

Based on direct cell counts generative times in hours were calculated (33). The division rate of cells was 0.54 day⁻¹ in TAP medium and 0.27 day⁻¹ in BG 11 (Table 1).

### Chlorophyll α and Carotenoids

For chlorophyll α and carotenoids, a one-ml sample was centrifuged into a pellet and sonicated with a Branson sonicator with a fine probe for one minute at 0° C. in ice cold 90% acetone. The contents were thoroughly mixed in a vortex mixer and extracted for 24 h at 4° C. in a refrigerator sufficient for complete extraction. The extracts were cleared by centrifugation in a Beckman CS 15R centrifuge, and their absorptions at 750 (blank), 664, 647, and 452 nm, were read in a Spectromax spectrofluorimeter that accommodates 96 well polypropylene NUNC plates.

The following equations were used to calculate pigment concentrations (μg ml⁻¹ culture):

Chl α=11.93D664−1.93D647(Vc/Vs) (34)

Carotenoid=3.86*D452(Vc/Vs) (35)

where Vc=volume of culture sample (ml) and Vs=Volume of extract (ml).

Quantitative measurement of fatty acids was performed by Avanti Polar Lipids, Inc. (www.Avantilipids.com) of fatty acid methyl ester (FAME) by gas chromatography with flame ionization (GC/FID) on 1.5 ml of extracted algae using 7-level calibration curves of FAME standards for C8-C24:1 compounds with a C15:1 as internal standard (36). Each sample was injected in triplicate. Standard deviation of the mean ranged between 0.01 and 0.04 when the mean total lipids were <6.0, and between 0.71 and 2.71 when the means were 15.35 to 22.64.

Two-way analysis of variance (ANOVA) was done on several variables using an EXCEL statistical package (37) to test significance of differences between treatments.

Wastewater Media

Filtered Albuquerque wastewater was enriched with TAP stock solutions nutrients (one ml each to 0.2 μm filtered liter of waste-water) and used either sterilized or unsterilized depending on the experimental design. The media were designated as: ST—Sterile Wastewater enriched with 1% TAP; NST—Non-sterile Wastewater enriched with 1% TAP; WWS—Sterile Wastewater; WWNS—Nonsterile wastewater.

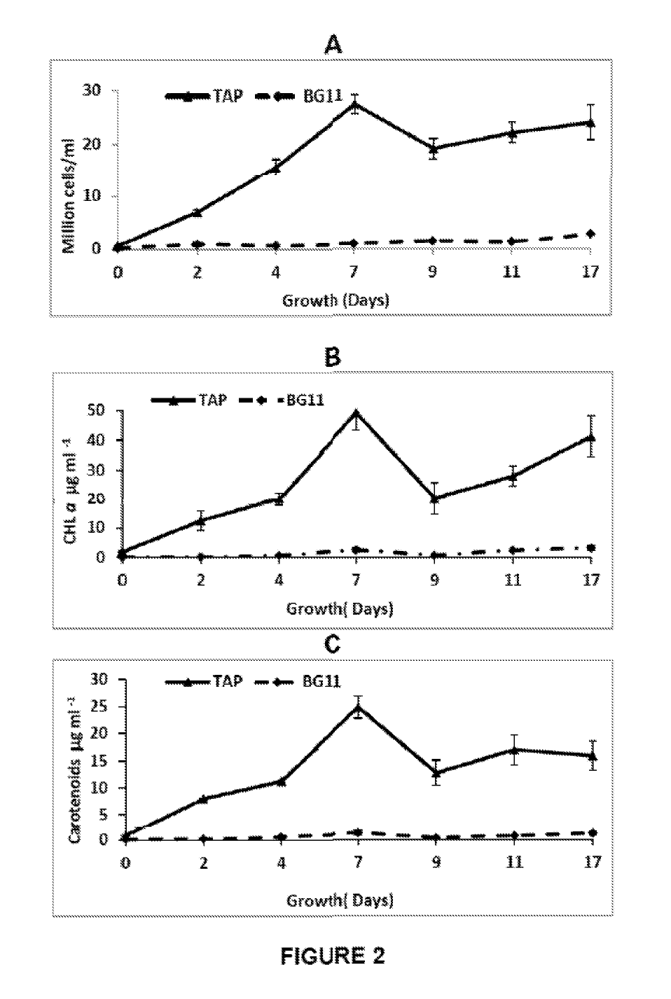

Algal cells grew readily in TAP medium and reached peak biomass levels (27.4×10⁶ cells ml⁻¹ (FIG. 2 A, Table 1), yielding 49.11 μg chlorophyll α ml⁻¹ (FIG. 2 B, Table 1) and 24.93 μg carotene ml⁻¹ on the 7^{th} day (FIG. 2 C, Table 1). Cells grew exponentially, reached a peak and subsequently decreased. Biomass levels were significantly low in BG11 medium. The division rate of cells was 0.54 day⁻¹ in TAP medium and 0.27 day⁻¹ in BG 11 (Table 1).

### Indoor Cultures in Wastewater

Cultures raised in the laboratory at 24±1° C. at continuous 132-148 μmol m⁻²s⁻¹ light in sterile wastewater enriched with 1% TAP supported good growth and yielded 10×10⁶ cells ml⁻¹, 17.6 μg chlorophyll α ml⁻¹, and 7.42 μg carotene ml⁻¹ (Table 1). The division rate was 0.24 day⁻¹ (Table 1). Growth in non-sterile wastewater, although enriched with 1% TAP, was 5.39×10⁶ cells ml⁻¹, yielding 6.79 μg chlorophyll α ml⁻¹, and 3.69 μg carotene ml⁻¹. However growth was high in unenriched sterile wastewater 10.18×10⁶ cells ml⁻¹, yielding 12.08 μg chlorophyll α ml⁻¹, and 7.64 μg carotene ml⁻¹ (Table 1), higher than in unenriched, nonsterile wastewater that has 4.33×10⁶ cells ml⁻¹, and yields 11.04 μg chlorophyll α ml⁻¹ and 5.56 μg carotene ml⁻¹ (Table 1).

Indoor wastewater cultures had more pigments per cell (range of 2.88 pg cell⁻¹ chlorophyll α to 3.43 pg cell⁻¹ chlorophyll α and 1.52 pg cell⁻¹ to 1.75 pg cell⁻¹ carotene compared to those grown either outdoors or in TAP or BG11 media (Table 1).

### Outdoor Cultures in Wastewater

Growth of cultures raised on the terrace of a building under harsh ambient conditions of light (1400-1600 watts) and temperature (˜40° C.) favorably compared to that of cultures raised indoors. The cultures raised under these harsh ambient conditions produced a biomass yielding 10.41×10⁶ cells ml⁻¹, 8.92 μg ml⁻¹ chlorophyll α, and 4.18 μg ml⁻¹ carotene (Table 1) in sterile wastewater enriched with 1% TAP; with a division rate of 0.24 day⁻¹. In unenriched sterile wastewater peak biomass was 8.81×10⁶ cells ml⁻¹, yielding 5.82 μgml⁻¹ chlorophyll α and 4.49 ml⁻¹ carotene (Table 1) with a cell division rate of 0.19 day⁻¹. Corresponding numbers for unenriched nonsterile wastewater cultures were 5.08×10⁶ cells ml⁻¹ biomass, yielding 5.41 μgml⁻¹ chlorophyll α, and 3.02 μgml⁻¹ carotene with a division rate of 0.14 day⁻¹ (Table 1).

### Analysis of Variance

Results of two-way analysis of variance (Table 2) showed that statistically significant differences existed in the biomass levels depending on the medium utilized. For example cultures grown in the defined TAP medium yielded higher levels of cells, biomass, chlorophyll α, and carotene cell⁻¹, than those in BG11 medium. Cultures grown in sterilized wastewater enriched with 1% TAP nutrients had significantly higher cell densities, chlorophyll α and carotene than those in similar media but unsterilized.

Production of biomass, i.e., cells, chlorophyll α and carotene in cultures grown indoors and outdoors in ST (Sterile medium enriched with 1% TAP), was significantly higher than in cultures grown in NST medium (non-sterile medium enriched with 1% TAP), WWS (sterile wastewater) and WWNS (nonsterile wastewater). However differences in chlorophyll α levels in cultures raised in non-sterile wastewater enriched with 1% TAP (NST) and in non-sterile wastewater (WWNS) were not statistically significant.

### Changes in Pigment Levels

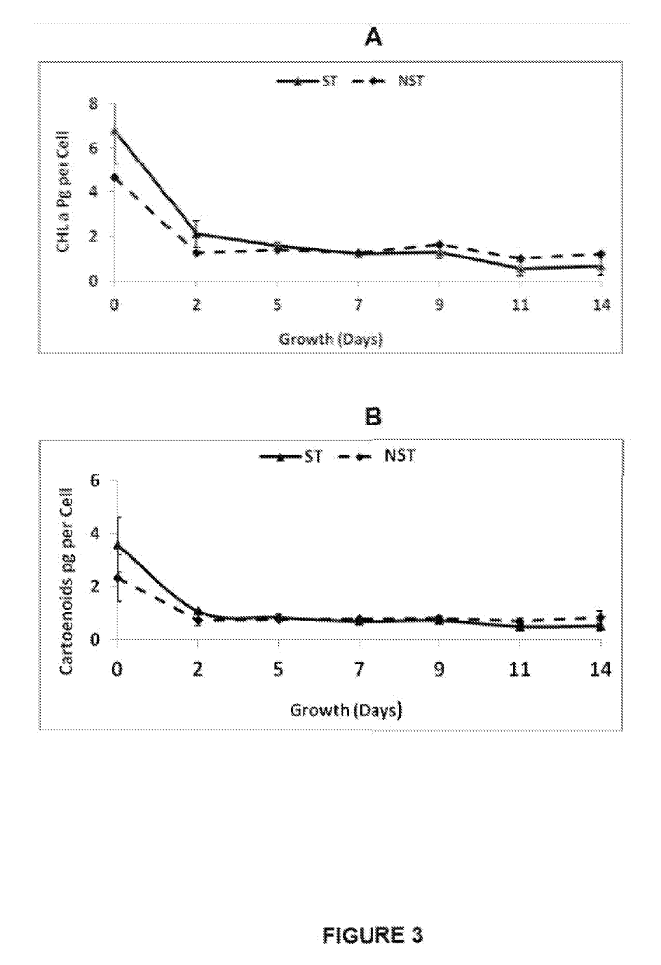

A feature of interest is the high initial levels of cellular chlorophyll α, and carotene and their gradual decrease with time (FIG. 3) in all cultures. For example cellular chlorophyll α levels (FIG. 3A) in cultures grown indoors were 0.67 pg cell⁻¹ (ST), 4.62 pg cell⁻¹ (NST), 3.26 pg cell⁻¹ (WWS) and 2.76 pg cell⁻¹ (WWNS) and the corresponding cellular carotene values were 3.58 pg cell⁻¹ (ST), 2.33 pg cell⁻¹ NST), 1.64 pg cell⁻¹ (WWS) and 1.52 pg cell⁻¹ (WWNS) (FIG. 3B).

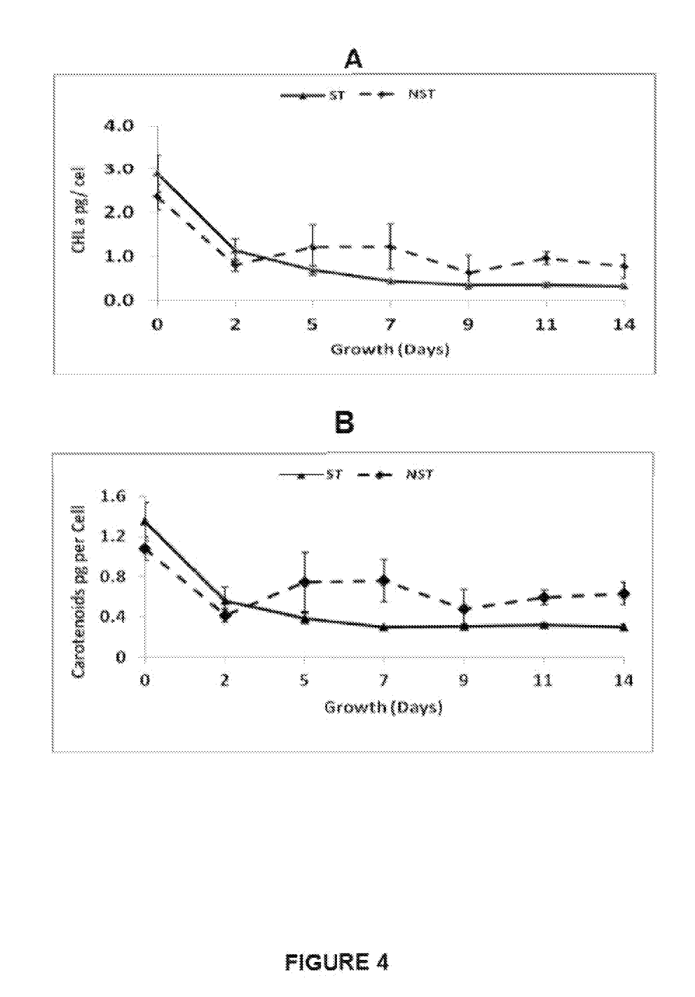

In outdoor cultures the initial cellular chlorophyll α levels (FIG. 4 A) were 2.89 pg cell⁻¹ (ST) 2.35 pg cell⁻¹ (NST), 4.83 pg cell⁻¹ (WWS) and 4.43 pg cell⁻¹ (WWNS). Corresponding carotenes were 1.35 pg cell⁻¹ (ST) 1.08 pg cell⁻¹ (NST), 0.93 pg cell⁻¹ (WWS) and 0.95 pg cell⁻¹ (WWNS). By day 14 the chlorophyll α decreased to about 14% to 85% in both indoor and outdoor cultures. Decreases in carotenes varied between 14% and 85% in indoor cultures and 22% to 63% in outdoor cultures (FIG. 4 B). Carotene also decreased and ranged from 36% to 85% in indoor cultures and 51% to 66% in outdoor cultures.

### Discussion

Our results show that microalgal extremophiles native to New Mexico can be brought into wastewater culture. Scenedesmus species Novo studied here is especially suited for mass cultivation and for utility in biotechnology. This alga is cultivable in wastewater and under the harsh ambient light and temperature conditions of semiarid regions such as Albuquerque, N. Mex. Its production is cost-effective, an important consideration in biotechnology applications. Biomass levels of our outdoor cultures were high (10.41×10⁶ cells ml⁻¹, yielding 8.92 μg chlorophyll α ml⁻¹, and 4.18 μg carotene ml⁻¹), and division rates (8) compared well with those obtained on cultures raised under measurable controlled, less severe conditions of temperature and light. Harvesting the algal biomass is also simple and cost-effective as our cultured cells settle readily to the bottom and separation does not require centrifugation, flocculation, or utilization of other energy-intensive methods.

A few investigators have studied the lipid as percent dry weight of cultured algae (Table 4); our algal cells had a range of 15-85% (Table 3, Table 4) compared 0.1 to 75% reported on several species (Table 4). Several studies reported potential for sustaining algal blooms in water enriched with wastewater from municipal sewage, agriculture and industrial sources and total lipids that varied between 9 and 29% of dry weight (9). Total lipids in Chlamydomonas reinhardtii were 25.25% dry weight (10); 17.85% in Botryococcus braunii (11), 9-13.6% in Chlorella ponds enriched with dairy manure (12), and 14% to 29% in mixed algae cultures originally isolated from local wastewater treatment ponds (13). Because of their high-value for biofuel, nutraceuticals and pharmaceuticals, carotenoids and lipids from microalgae have been studied, with most investigators reporting these values as percent of cell dry weight, lipid production as mgl⁻¹d⁻¹, gl⁻¹d⁻¹, and g m⁻²d⁻¹ (1, 14, 9, 15, 16, 17, 18). Preliminary analyses of lipids on our algal slurries (Table 3) showed that lipid yield was initially high, reaching a peak (94.3 pg cell⁻¹) following 8 days of growth.

Cellular carotene in our algal cells ranged from 0.95 to 3.58 pg cell⁻¹ and compared favorably with carotene yields (Table 5) for Dunaliella salina (19) or D. salina, D. bardawil and 18 strains of microalgae isolated from tropical waters of the Bay of Bengal (20).

We have successfully brought the extremophile alga Scenedesmus species Novo, native to New Mexico, into culture.

Sequencing of the new Jemez alga was completed utilizing three different primers to completely sequence the 18S rDNA (32) and the data were used to assemble the contig. The 18S rDNA sequence of Scenedesmus species Novo [SEQ ID NO:1] is shown in the Sequence Listing at the end of this Specification.

Sequencing of the Jemez alga showed that it is most closely related to G24 (but less than 99% homologous to G4, and more distant from Scenedesmus abundans and S. communis.

In the defined TAP medium, under controlled conditions of temperature and light, high levels of biomass (cells), chlorophyll α and carotene and division rates were sustained. Further this extremophile alga grew well in wastewater under controlled conditions of temperature and light and under harsh ambient temperatures and light as well. An added advantage of our cultures is the settlement of cells readily to the bottom which makes their harvesting simple, and cost effective.

Cellular lipids in our cultures are the highest reported for microalgae. Lipids in cultures attained their peak (94.3 pg cell⁻¹) in a relatively short time, remained high and contributed between 57% and 85% of cell weight. Carotenoids were also high (0.95-3.58 pg cell⁻¹) and compared favorably with those obtained on 18 strains of microalgae isolated from the tropical waters of the Bay of Bengal.

Scenedesmus species Novo grows rapidly under harsh climatic conditions and in wastewater. Through biochemical manipulation lipid and carotene synthesis can be regulated in algae. This involves imposing a physiological stress such as nutrient starvation to channel metabolic processes towards accumulation of bioactive compounds. To enhance yield of microalgal biomass, micronutrients such as selenium, boron and iron can be optimized, along with temperature and light.

Antimicrobial Activity of Extracts of Scenedesmus novo

S. novo cells are grown at 22° C. under continuous light conditions for 10 days to achieve a dense culture. Final volume of culture is 2.0 liters. Cells are centrifuged and cell pellets are subjected to lysis using sonication in the setting of proteinase K and bath temperatures of 4° C. to avoid inactivation of proteins. Cell lysate is decanted and tested in antimicrobial screening assays against a control extract prepared from a Chlorella species.

Antimicrobial assays are conducted using turbidity assessments for Minimum Inhibitory Concentrations. Target species of bacteria include E. coli, S. aureus, K pneumonia and P. vulgaris. In all cases, cell lysates of S. novo exhibit inhibition of growth of bacteria at 12 hours in a 96-well plate assay.

Antiparasite assays are conducted as above using 2 target organisms: Trypanosoma cruzi strain “Y” and Leishmania donovani. Cell lysates of S. novo inhibit parasite growth at 24 hours.

Antifungal assays are conducted with Candida albicans and inhibition of fungal growth in a broth assay is observed at 24 hours with cell lysates of S. novo.

TABLE 1
Maximum mean values of cell numbers, chlorophyll α, and carotenoids with standard
deviations and day of attainment, in cultures of Scenedesmus sp. Novo

| | | Cells | Chl α | Carotene | Chl α | Carotene | Chl α: | K cell |
|---|---|---|---|---|---|---|---|---|
| Growth | | 10⁶ ml⁻¹ | μg ml⁻¹ | μg ml⁻¹ | pg cell⁻¹ | pg cell⁻¹ | Carotene | div. d⁻¹ |
| 1. TAP & | TAP | 27.42 | 49.11 | 24.93 | 3.1 | 1.43 | 2.6 | 0.54 |
| BG11 | S.D | 1.83 | 6.02 | 1.3 | 0.91 | 0.4 | 0.2 | |
| | Day | 7 | 7 | 7 | 0 | 0 | 17 | |
| | BG 11 | 2.7 | 3.18 | 1.3 | 2.83 | 1.41 | 3.44 | 0.27 |
| | S.D | 0.13 | 0.88 | 0.4 | 1.17 | 0.64 | 0.43 | |
| | Day | 17 | 17 | 7 | 7 | 7 | 11 | |
| 2. Indoors | ST | 10 | 17.6 | 7.42 | 6.79 | 3.58 | 2.42 | 0.24 |
| | S.D | 0.26 | 0.73 | 1.36 | 2.22 | 1.04 | 0.46 | |
| | Day | 9 | 2 | 2 | 0 | 0 | 2 | |
| | NST | 5.39 | 6.79 | 3.69 | 4.62 | 2.33 | 2.33 | 0.25 |
| | S.D | 0.14 | 2.99 | 0.52 | 0.75 | 0.39 | 0.14 | |
| | Day | 11 | 5 | 11 | 0 | 0 | 9 | |
| | WW S | 10.18 | 12.08 | 7.64 | 3.43 | 1.75 | 2.32 | 0.15 |
| | S.D | 1.69 | 2.24 | 1.55 | 1.51 | 0.75 | 0.5 | |
| | Day | 5 | 2 | 14 | 2 | 2 | 9 | |
| | WW NS | 4.33 | 11.04 | 5.56 | 2.88 | 1.52 | 2.02 | 0.11 |
| | S.D | 0.36 | 1.51 | 0.38 | 0.68 | 0.45 | 0.12 | |
| | Day | 14 | 11 | 14 | 0 | 0 | 11 | |
| 3. Out doors | ST | 10.41 | 8.92 | 4.18 | 2.89 | 1.35 | 2.14 | 0.24 |
| | S.D | 2.59 | 0.81 | 0.5 | 0.18 | 0.19 | 0.06 | |
| | Day | 5 | 0 | 0 | 0 | 0 | 0 | |
| | NST | 5.65 | 7.13 | 3.65 | 2.35 | 1.08 | 2.17 | 0.28 |
| | S.D | 0.54 | 0.45 | 1.63 | 0.45 | 0.11 | 0.06 | |
| | Day | 14 | 0 | 5 | 0 | 0 | 0 | |
| | WW S | 8.81 | 5.82 | 4.49 | 2.01 | 0.93 | 2.22 | 0.19 |
| | S.D | 0.38 | 2.25 | 1.64 | 0.18 | 0.05 | 0.03 | |
| | Day | 14 | 14 | 14 | 0 | 0 | 0 | |
| | WW NS | 5.08 | 5.41 | 3.02 | 2.19 | 0.95 | 2.24 | 0.14 |
| | S.D | 0.3 | 0.83 | 0.32 | 0.29 | 0.04 | 0.08 | |
| | Day | 14 | 5 | 14 | 0 | 0 | 0 | |

TABLE 2
Summary of results on two way analysis of variance (d.f 41, F critical = 2.44).

| Growth | Source* | Variable | F value | probability | Significance |
|---|---|---|---|---|---|
| 1. Defined | TAP-BG11 | cells | 86.68 | 8.97 E−17 | Highly |
| media | | Chl α | 45.20 | 3.94 E−13 | significant |
| | | carotene | 50.89 | 8.85 E−14 | |
| | | Chl α/cell | 8.11 | 3.90 E−05 | |
| | | Carotene/cell | 5.50 | 0.0001 | |
| 2. Indoor | ST- NST | cells | 39.29 | 2.24 E−12 | |
| cultures | ST-WW S | | 55.89 | 2.68 E−14 | |
| | NST-WW NS | | 32.79 | 2.03 E−11 | |
| | WW S-WW NS | | 46.06 | 3.12 E−13 | |
| | ST- NST | Chl α | 4.68 | 0.002 | |
| | ST-WW S | | 8.459 | 2.76 E−05 | |
| | NST-WW NS | | 6.996 | 0.0001 | |
| | WWS-WW NS | | 12.05 | 1.15 E−06 | |
| | ST- NST | carotene | 3.144 | 0.017 | |
| | ST-WW S | | 8.02 | 4.294 E−05 | |
| | NST-WW NS | | 11.35 | 2.03- E−06 | |
| | ST-WW NS | | 20.0 | 6.12 E−09 | |
| 3. Outdoor | ST-NST | cells | 19.11 | 1.005 E−08 | |
| cultures | ST-WW S | | 14.53 | 0.0001 | |
| | NST-WW NS | | 4.35 | 0.003 | |
| | ST-WW NS | | 87.65 | 7.75 E−17 | |
| | ST- NST | Chl α | 10.68 | 3.55 E−06 | |
| | ST-WW S | | 6.648 | 0.00002 | |
| | NST-WW NS | | 2.32 | 0.060 | Not significant |
| | ST-WW NS | | 1.93 | 0.110 | Not significant.S |
| | ST- NST | carotene | 3.37 | 0.012 | Highly significant |
| | ST-WWS | | 3.059 | 0.019 | |
| | NST-WW NS | | 3.67 | 0.008 | |
| | ST-WW NS | | 6.38 | 0.0002 | |

*ST—Sterile wastewater enriched with 1% TAP; NST—Non-sterile wastewater enriched with 1% TAP WWS—Sterile Wastewater; WWNS—Non-sterile wastewater.

TABLE 3
Cell numbers and lipids in Scenedesmus sp
Novo in Wastewater enriched with TAP nutrients.

| | | | pg | pg lipid/ | |
|---|---|---|---|---|---|
| Growth | | Cells | lipid/ | pg cell | Lipid % |
| (Days) | Temp. and light | 10⁶/ml | cell | dry wt | cell dry wt |
| 2 | 24 ± 1° C., continuous | 0.32 | 66.6 | 0.60 | 60 |
| 3 | 132-148 μmol photons | 0.79 | 63 | 0.57 | 57 |
| 6 | m⁻² s⁻¹ | 1.97 | 76.8 | 0.69 | 69 |
| 8 | | 2.4 | 94.3 | 0.85 | 85 |
| 9 | | 2.7 | 82.5 | 0.75 | 75 |
| 2 | ~40° C. and Daylight | 0.3 | 16.7 | 0.15 | 15 |
| 4 | 6524-7360 μmol | 0.62 | 20.2 | 0.18 | 18 |
| 5 | photons m⁻² s⁻¹ | 0.78 | 36.6 | 0.33 | 33 |
| 7 | | 0.82 | 29.2 | 0.26 | 26 |
| 9 | | 0.85 | 20.8 | 0.19 | 19 |

TABLE 4
Lipids (pg cell⁻¹) in selected microalgal cultures.

| | Growth | Lipid | Lipid pg/pg | |
|---|---|---|---|---|
| Taxa | (Days) | pg/cell | cell dry wt | Reference |
| Scenedesmus sp Novo | 1-9 | 63 to 94.3 | 0.15 to 0.85 | Present study |
| Scenedesmus sp obliquus | | | 0.06-0.184 | (18) Chen et al. 2011 |
| S. obliquus | | | 0.06-0.12 | (21) Mandal and Mallick 2009 |
| S. obliquus | | | 0.128 | (22)Silva et al. 2010 |
| | | | 0.11-0.55 | (23) Gouveia and Oliveira 2009 |
| Chlorella vulgaris | | | 0.14-0.55 | (23) Gouveia and Oliveira 2009 |
| Chlorella sps. | | | 0.34-0.67 | (18) Chen et al. 2011 |
| Chlorella prothecoides | | | 0.11-0.23 | (18) Chen et al. 2011 |
| Dunaliella tertiolecta | | | 0.678 | (18) Chen et al. 2011 |
| Neochloris oleabundans | | | 0.35-0.65 | (23) Gouveia and Oliveira 2009 |
| N. oleabundans | | | 0.165 | (22) Silva et al. 2010 |
| Botryococcus braunii | | | 0.5 | (24) Kojima and Zhang 1999 |
| Botryococcus braunii | | | 0.25-0.75 | (1) Chisti 2007 |
| Several algae | | | 0.06-0.678 | (18) Chen et al. 2011 |
| 8 species | | | 0.05-0.63 | (16) Mata et al. 2010 |
| 21 species | | | 0.05-0.678. | (18) Chen et al. 2011 |
| 18 strains tropical algae | 16 | 0.22-14.77 | | (20) Keerthi pers. com |
| | 21 | 0.07-44.85 | | |
| D. salina | 16 | 0.21-3.45 | | |
| | 21 | 0.04-44.85 | | |
| D. bardawil | 16 | 1.25-12.78 | | |
| | 21 | 0.06-0.30 | | |
| D. tertiolecta | 16 | 0.25-1.97 | | |
| | 21 | 0.14-22.16 | | |
| D. parva | 16 | 0.76-14.77 | | |
| | 21 | 0.29-0.46 | | |
| Nannochloropsis sp. | | 0.07-0.35 | 0.02-0.04 | (15) Huerlimann et al. 2010 |
| Isochrysis sp. | | 1.16-4.93 | 0.02-0.03 | |
| Tetraselmis sp. | | 4.37-29.11 | 0.008-0.13 | |
| Rhodomonas sp. | | 0.79-12.27 | 0.001-0.017 | |
| Nannochloropsis sp | | | 0.22-0.60 | (17) Rodolphi et al. 2009 |

TABLE 5
Carotenoids (pg cell⁻¹) in selected microalgae.

| | Media | Caroten | |
|---|---|---|---|
| Alga | NaCl % | pg cell⁻¹ | Reference |
| Scenedesmus | Fresh | 0.95-3.58 | Present study |
| species Novo | water | | |
| Dunaliella salina | | 0.35-1.77 | (19) Mendoza et al. 2008 |
| Nannochloropsis | | 0.016 | (25) Forzan et al. 2007 |
| galitana | | | |
| Haematococcus | | 25 | (26) Cifuentes et al. 2003 |
| pluvialis | N₂ | 8-15 | |
| | normal | | |
| | N₂ | 10.3-25 | |
| | deprived | | |
| 18 strains of | | 0.24 to 4.75 | (20)Keerthi pers. com |
| microalgae | | | |
| Dunaliella | 10 | 0.67-27.53 | (20) Keerthi pers. com |
| bardawil | 12.5 | 0.49-2.07 | |
| | 15 | 0.32-14.07 | |
| | 20 | 1.67-3.79 | |
| | 25 | 0.61-7.92 | |
| | 30 | 0.57-7.28 | |
| D. salina | 10 | 0.3-1.61 | (20) Keerthi pers. com |
| | 12.5 | 0.3-1.89 | |
| | 15 | 0.34-1.69 | |
| | 20 | 0.36-1.77 | |
| | 25 | 0.38-1.85 | |
| | 30 | 0.27-1.61 | |
| D. salina | | 1.65-8.28 | (27) Pisal and Lele 2005 |

### REFERENCES FOR BACKGROUND OF THE INVENTION AND EXAMPLE 2

1. Ravi V. Durvasula, D. V. Subba Rao and V. S. Rao, 2013. Microalgal Biotechnology: Today's (Green) Gold Rush. Ch. 13, pp. 201-107, in. (Ed. F. Bux) Biotechnological Applications of Microalgae, CRC Press, Boca Raton, London, New York.
2. Chisti Y (2007) Biodiesel from microalgae. Biotechnol Adv 25: 294-306.
3. Haag A L (2007) Algae Bloom again. Nature 447: 520-521.
4. Weyer K M, et al. (2010) Theoretical maximum Algal Oil Production. Bioenerg. Res. 3: 204-213.
5. International Energy Agency Report http://www.iea.org/journalists/index.asp
6. Alabi A O (2009) Microalgal technologies and processes for biofuels/bioenergy production in British Columbia. The British Columbia Innovation Council 1-74 pp.
7. Liska A J, et al. (2004) Enhanced Photosynthesis and Redox Energy Production Contribute to Salinity Tolerance in Dunaliella as Revealed by Homology-Based Proteomics. Plant Physiology 136:2806-2817.
8. Subba Rao D V, et al. (2005) Growth and photosynthetic rates of Chlamydomonas plethora and Nitzschia frustula cultures isolated from Kuwait Bay, Arabian Gulf, and their potential as live algal food for tropical mariculture. Marine Ecology, 26: 63-71.
9. Subba Rao D V (2009) Cultivation, Growth Media, Division rates and applications of Dunaliella species. Pp 45-90. In: Ben-Amotz A, Polle J E W, Subba Rao D V, editors. Alga Dunaliella Biodiversity, Physiology, Genomics and biotechnology. Enfield: Science Publishers; 2009.
10. Pittman J K, et al. (2010) The potential of sustainable algal biofuel production using wastewater resources. Bioresource Technology 102: 17-25.
11. Kong Q X, et al. (2010) Culture of microalgae Chlamydomonas reinhardtii in wastewater for biomass feedstock production, Appl. Biochem. Biotechnol. 160: 9-18.
12. Orpez R, et al. (2009) Growth of the microalga Botryococcus braunii in secondarily treated sewage, Desalination 246: 625-630.
13. Wang Y C, et al. (2010) Anaerobic digested dairy manure as a nutrient supplement for cultivation of oil-rich green microalgae Chlorella sp, Bioresour. Technol. 101: 2623-2628.
14. Woertz I, et al. (2009) Algae Grown on Dairy and Municipal Wastewater for Simultaneous Nutrient Removal and Lipid Production for Biofuel Feedstock. Jour. Envi. Eng. © ASCE/November 135: 1115-1122.
15. Harun R, et al. (2010) Bioprocess engineering of microalgae to produce a variety of consumer products. Renewable and Sustained Energy reviews 14: 1037-47.
16. Huerlimann R, et al. (2010) Growth, lipid content, productivity, and fatty acid composition of tropical microalgae for scale-up production. Biofuels and Environmental Biotechnology DOI 10. 1002/bit.22809
17. Mata T M, et al. (2010) Microalgae for biodiesel production and other applications: A review. Renewable and Sustainable Energy reviews. 14: 217-32.
18. Rodolfi L, et al. (2008). Microalgae for Oil: Strain selection, induction of lipid synthesis and outdoor mass cultivation in a low-cost photobioreactor, Biotechnology and Bioengineering. 102: 100-112.
19. Chen C Y, et al. (2011). Cultivation, photobioreactor design and harvesting of microalgae for biodiesel production: A critical review. Bioresource Technology 102: 71-81.
20. Mendoza H, et al. (2008) Characterization of Dunaliella salina strains by flow cytometry: a new approach to select carotenoid hyperproducing strains Electronic Journal of Biotechnology ISSN: 0717-3458, 11: 2-13.
21. Keerthi et al. 2012 personal communication
22. Mandal S, Mallick N (2009) Microalga Scenedesmus obliquus as a potential source for biodiesel production. Appl Microbiol Biotechnol 84: 281-91.
23. Silva T L, ert al. (2010) Oil Production Towards Biofuel from Autotrophic Microalgae Semicontinuous Cultivations Monitorized by Flow Cytometry. Applied Biochemistry and Biotechnology 159: 568-578, DOI: 10.1007/s 12010-008-8443-5.
24. Gouveia L, Olievera A C (2009) Microalgae as a raw material for biofuel production. Journal of Industrial Microbiology and Biotechnology. 36: 269-74. DOI:10. 1007/s 10295-008-0495-6.
25. Kojima E, Zhang K (1999) Growth and hydrocarbon production of microalga Botryococcus braunii in bubble column photobioreactors. Journal of Bioscience and Bioengineering: 811-815.
26. Forján E, et al. (2007) Enhancement of carotenoid production in Nannochloropsis by phosphate and sulphur limitation. pp 356-364 in Communicating Current Research and Educational Topics and Trends in Applied Microbiology. (Ed). A. Méndez-Vilas.
27. Cifuentes A S, et al. (2003) Optimization of biomass, total carotenoids and astaxanthin production in Haematococcus pluvialis, Flotow strain Steptoe (Nevada, USA) under laboratory conditions. Biol. Res. 3 6: 343-357.
28. Pisal D S, Lele S S (2005) Carotenoid production from microalga, Dunaliella salina. Ind Jour Biotech. 4: 476-483.
29. Guillard R R L, Ryther J H (1962) Studies of marine planktonic diatoms. I. Cyclotella nana Hustedt and Detonula confervacea Cleve. Can. J. Microbiol. 8: 229-239.
30. Stanier R Y, et al. (1971) Purification and properties of unicellular bluegreen algae (Order Chroococcales). Bacteriol. Rev. 35: 171-205.
31. Gorman D S, Levine R P (1965) Cytochrome f and plastocyanin: Their sequence in the photosynthetic electron transport chain of Chlamydomonas reinhardttii. Proc. Natl. Acad. Sci. USA 5: 1665-1669.
32. Hutner S H, et al. (1950) Some approaches to the study of the role of metals in the metabolism of microorganisms. Proc. Am. Philos. Soc. 94: 152-170.
33. GenBank: HQ900842.1
34. Furnas M (2002) Measuring the growth rates of phytoplankton in natural populations. In: Subba Rao D. V. (Ed), Pelagic Ecology Methodology. A. A. Balkema Publishers Lisse/Tokyo, 221-249.
35. Jeffrey S W, Humphrey G F (1975) New spectrophotometric equations for determining chlorophylls a, b, c₁ and c₂ in higher plants, algae and natural phytoplankton. Biochem. Physiol. Pflanzen. 167: 191-194.
36. Borowitzka M J, Siva C J (2007) The taxonomy of the genus Dunaliella (Chlorophyta, Dunaliellales) with emphasis on the marine and halophilic species. Journal of Applied Phycology 19: 567-590.
37. www.AvantiLipidscom
38. Dytham C (1999) Choosing and using Statistics: A Biologist's Guide. Blackwell Sciences. 218 pp.
39. Nold, S. C., et al. (2010), “Eukaryal and archaeal diversity in a submerged sinkhole ecosystem influenced by sulfer-rich, hypoxic groundwater,” Great Lakes Res. 36, 366-375, GenBank Accession No. EU910612.

### Example 2

We enriched municipal waste water with 1% TAP (Gorman and Levine 1965) nutrients. This is probably the most widely-used medium at present for experimental work. The following stock solutions were used:
1. TAP salts
NH₄Cl 15.0 g
MgSO₄.7H₂O 4.0 g
CaCl₂.2H₂O 2.0 g
water to 1 liter
2. phosphate solution
K₂HPO₄ 28.8 g
KH₂PO₄ 14.4 g
water to 100 ml
3. Hunter's trace elements

To make the final medium, mix the following:
2.42 g Tris
25 ml solution #1 (salts)
0.375 ml solution #2 (phosphate)
1.0 ml solution #3 (trace elements)
1.0 ml glacial acetic acid
water to 1 liter

We have isolated an extremophile green alga Scenedesmus, from Soda Dam warm water springs, New Mexico. Whether grown in water enriched with 1% TAP nutrients or un-enriched, high levels of biomass could be sustained in sterilized or un-sterilized municipal wastewater. Under outdoor conditions (6524-7360 μmol photons m⁻² s⁻¹ and ˜40° C.) high levels of biomass (10.41×10⁶ cells ml⁻¹, 8.92 μg chl a ml⁻¹, and 4.18 μg carotene ml⁻¹) could be sustained. Under controlled conditions lipids in cells raised in TAP ranged from 63 to 94.3 pg cell⁻¹ and in outdoor wastewater 16.7 to 81.4 pg cell⁻¹ which are higher than those reported. In cultures raised in TAP medium lipid (% of cell dry weight) ranged from 57 to 85% compared to 15-74% in outdoor waste water which are also substantially higher than literature values. Total carotenoids ranged between 0.37 and 3.58 pg cell⁻¹ compared to 0.24-4.75 pg cell⁻¹ in literature.

Because of its amenability to produce high levels of microalgal biomass in wastewater under harsh ambient climatic conditions, and yield of high levels of lipids and carotenes, Scenedesmus species Novo has the potential to sustain biotechnological applications. Notably, the microalgae biomass can produce biodiesel (Christi 2007), bioethanol (Harun et al. 2010), biogas, and biohydrogen (Demirbas, 2010). and bio-oils. Since the novel alga can be cultured in wastewater, it has potential for bioremediation and production of valuable products. We recommend more isolations of several extremophile algal species native to New Mexico with a view to develop strategies for a viable bio-economy based on their mass cultivation.

### REFERENCES FOR EXAMPLE 2

Barclay, W. R., K M Meager, J R Abril 1994. Heterotrophic production of long chain omega-3 fatty acids utilizing algae and algae-like microorganisms. Journal of Applied Phycology, 6: 123-129.
Bashan, L. E. e Bashan, Y. 2010. Immobilized microalgae for removing pollutants: review of practical aspects. Bioresource Technology 2010; 101(6) 1611-27. http://dx.doi.org/10.10161j.biortech.2009.09.043.
Behrens P W, Thompson J M, Apt K, Pfeifer J W, Wynn J P, and Lippmeier J C. 2005. Production of high levels of DHA in microalgae using modified amounts of chloride and potassium. J Exp Mar Biol Eco1.197:91-99 doi:10.1016/0022-0981(95)00146-8.
Bennemann, J. 2013. Microalgae for biofuels and animal feeds. Energies 6; 5869-5886. Bennemann, J. R. 2008, NREL-AFOSR Workshop, Algal oil for jet fuel production; Arlington, 19 Feb. 2008.
Chini Zittelli, G., F. Lavista, A. Bastianini, L. Rodolfi, M. Vincenzini, M. R. Tredici. 1999. Production of eicosapentaenoic acid by Nannochloropsis sp. cultures in outdoor tubular photobioreactors. Journal of Biotechnology 70 (1999) 299-312.
Chisti, Y. 2007. Biodiesel from microalgae. Biotechnology Advances, v. 25, n. 3, p. 294-306, http://dx.doi.org/10.1016/j.biotechadv.2007.02.001.
Coutteau, P., and P. Sorgeloos. 1992. The use of algal substitutes and the requirement for live algae in the hatchery and nursery rearing of bivalve molluscs: an international survey. Journal of Shellfish Research 11: 467-476.
Day, J G., Edwards, A P. And Rogers G A. 1991. Development of an industrial-scale process for heterotrophic production of micro-algal mollusk feed. Bioresource Technol. 38: 245-249.
Demirbas, M. F. 2011. Biofuels from algae for sustainable development. Applied Energy 88:3473-3480. http://dx.doi.org/10.1016/j.apenergy.2011.01.059.
De Pauw, N., Morales, j. and Persoone, G. 1984. Mass culture of microalgae in aquaculture systems: progress and constraints. Hydrobiologia 116/117, 121-134.
De Pauw, N. and Persoone, G. 1988. Microalgae for aquaculture. In; Borowitzka, M. A. and Borowitzka, L. J. (Eds.) Mico-algal biotechnology. Cambridge University Press. Cambridge pp. 197-221.
De Swaaf, M., Rijk T C de, Eggink G. and Sijtsma, L. 1999. Optimisation of docosahexaenoic-acid production in batch cultivations by Crypthecodinium cohnii. J. Biotechnol. 70: 185-192.
Donaldson, J. 1991. Commercial production of microalgae at Coast Oyster Company. In: Rotifer and microalgae culture systems, Proceedings of a US-Asia Workshop, Honolulu, Hi., Jan. 28-31, 1991. Fulks, W. and K. L. Main (eds). The Oceanic Institute, Hawaii, USA, pp 229-236.
Glaude, R. M. and Maxey, J. E. 1994. Microalgal feeds for aquaculture. Journal of Applied Phycology, 6: 131-141, 1994.
Gorman D S, Levine R P 0.1965. Cytochrome f and plastocyanin: Their sequence in the photosynthetic electron transport chain of Chlamydomonas reinhardttii. Proc. Natl. Acad. Sci. USA 5: 1665-1669.
Harun, R. Boyin Liu, and M. K. Danquah. 2010. Microalgal biomass as a cellulosic fermentation feedstock for bioethanol production. Renewable and Sustainable Energy Reviews 2010. http://dx.doi.org/10.1016/j.rser.2010.07.071.
Jiang, L. Luo S, Fan X, Yang Z, Guo R. 2011. Biomass and lipid production of marine microalgae using municipal wastewater and high concentration of CO₂. Applied Energy 2011; 88(10) 3336-3341.http://dx.doi.org/10.1016/j.apenergy.2011.03.043.
Park, J. B. K., Craggs, R. and Shilton, A. S. 2011. Wastewater treatment high rate algal ponds for biofuel production. Bioresource Technology 2011; 102(1) 35-42. http://dx.doi.org/10.1016/j.biortech.2010.06.158
Posadas, E., Pedro-Antonio García-Encina, Anna Soltau, Antonio Dominguez, Ignacio Diaz, Raúl Muñoz, 2013. Carbon and nutrient removal from centrates and domestic wastewater using algal-bacterial biofilm bioreactors. Bioresource Technology, 139: 50-58.
Quilliam, R. S., Melanie A. van Niekerk, David R. Chadwick, Paul Cross, Nick Hanley, Davey L. Jones, Andy J. A. Vinten, Nigel Willby, and David M. Oliver, 2015. Can macrophyte harvesting from eutrophic water close the loop on nutrient loss from agricultural land? Journal of Environmental Management, 152: 210-217.
Saikumar, C. 2014. Bioremediation of wastewater using microalgae, Ph.D thesis, University of Dayton, 2014, 212 pages.
Sirakov, I. N. and K. N. Velichkova 2014. Bioremediation of wastewater originate from aquaculture and biomass production from microalgae species—Nannochloropsis oculata and Tetraselmis chuii. Bulgarian Journal of Agricultural Science, 20 (No 1) 2014, 66-72.
Sommers, L. E. 1977. Chemical composition of sewage sludges and analysis of their potential use as fertilizers. Jour. Envir. Quality 6: 225-232.
Vidali, M., (2001) Bioremediation An overview, Pure Appl. Chem., Vol. 73, No. 7, pp 1163-1172.
Walsh, D. T., C. A. Withstandley, R. A. Kraus, and E. J. Petrovits. 1987. Mass culture of selected marine microalgae for the nursery production of bivalve seed. Journal of Shellfish Research Vol. 6: 71-77.

All publications referred to herein are incorporated herein by reference to the extent not inconsistent herewith.

Numerical ranges mentioned herein specifically include all numbers to two decimal places that fall between the stated end points of the ranges.

It will be understood that although specific organisms, reagents, method steps and process conditions have been provided herein, equivalents of these are considered to be within the scope of the appended claims.

1-13. (canceled)

14. A method for culturing and harvesting extremophilic microalgae comprising:
preparing a growth composition comprising said extremophilic microalgae and water comprising nutrients capable of enhancing growth of said microalgae;
allowing said microalgae to proliferate in said composition at room temperature or under ambient outdoor conditions comprising intervals of ambient temperatures of at least about 40° C. and ambient light of up to about 1,400 to about 1,600 watts;
dewatering said composition and recovering an algal biomass comprising said microalgae and less than about 5% water content.

15. The method of claim 14 wherein said dewatering is performed in a micro solid-liquid separation system.

16. The method of claim 14 wherein said extremophilic microalgae is Scenedesmus species Novo.

17. The method of claim 14 wherein said growth composition comprises Scenedesmus species Novo and wastewater.

18. The method of claim 17 wherein said wastewater is sewage/municipal wastewater.

19. The method of claim 14 wherein said nutrients suitable for enhancing growth are selected from the group consisting of TAP medium components, selenium, boron, iron and mixtures thereof.

20. A method of inhibiting growth of a microorganism comprising contacting cells of said microorganism with an extract of Scenedesmus species Novo.

21. The method of claim 19 wherein said microorganism is selected from the group consisting of bacteria, viruses, parasites, and fungi.

22. A method of treating wastewater comprising:
preparing a growth composition comprising an extremophilic microalgae and said wastewater wherein said wastewaster comprises nutrients capable of enhancing growth of said microalgae;
allowing said microalgae to proliferate in said composition at room temperature or under ambient outdoor conditions comprising intervals of ambient temperatures of at least about 40° C. and ambient light of up to about 1,400 to about 1,600 watts; and
dewatering said composition and recovering an algal biomass comprising said microalgae and less than about 5% water content.

23. The method according to claim 22 wherein said extremophilic microalgae is Scenedesmus species Novo.

24. The method according to claim 22 wherein said wastewater is sewage/municipal wastewater.

25. The method according to claim 22 wherein said wastewater is a combination of sewage/municipal wastewater and industrial wastewater.

26. A method of treating sewage and/or wastewater to promote bioremediation, said method comprising
preparing a growth composition comprising an extremophilic microalgae and said sewage and/or wastewater wherein said sewage and/or wastewaster comprises nutrients capable of enhancing growth of said microalgae;
allowing said microalgae to proliferate in said composition at room temperature or under ambient outdoor conditions comprising intervals of ambient temperatures of at least about 40° C. and ambient light of up to about 1,400 to about 1,600 watts;
testing said sewage and/or wastewater to determine the level of pollutants and
recovering an algal biomass comprising said microalgae from said sewage and/or wastewater when the level of pollutants in said sewage and/or said wastewater reaches a desired level.

27. The method according to claim 26 wherein said extremophilic microalgae is Scenedesmus species Novo.

28. The method according to claim 22 wherein said wastewater is sewage/municipal wastewater.
